(19) United States
(12) Patent Application Publication
(10) Pub. No.: US 2017/0241851 A1
(43) Pub. Date: Aug. 24, 2017

(54) PRESSURE SENSOR, PRESSURE SENSOR SYSTEM, MICROPHONE, BLOOD PRESSURE SENSOR AND TOUCH PANEL

(71) Applicant: KABUSHIKI KAISHA TOSHIBA, Tokyo (JP)

(72) Inventors: Shotaro BABA, Tokyo (JP); Yoshihiko FUJI, Kawasaki (JP); Kei MASUNISHI, Kawasaki (JP); Michiko HARA, Yokohama (JP); Akiko YUZAWA, Kawasaki (JP); Shiori KAJI, Kawasaki (JP); Tomohiko NAGATA, Yokohama (JP); Yoshihiro HIGASHI, Komatsu (JP); Kenji OTSU, Yokohama (JP); Kazuaki OKAMOTO, Yokohama (JP)

(21) Appl. No.: 15/262,795

(22) Filed: Sep. 12, 2016

(30) Foreign Application Priority Data
Feb. 18, 2016 (JP) 2016-029093

(51) Int. Cl. G01L 9/00; A61B 5/021; A61B 5/00; G06F 3/041

(52) U.S. Cl. CPC G01L 9/0041; G06F 3/0414; A61B 5/02141; A61B 5/6801; H04R 19/04

(57) ABSTRACT

According to one embodiment, a pressure sensor includes a film portion, a sensor unit, and a structure body. The film portion has a front surface and is deformable. The sensor unit includes a plurality of sensing elements arranged along the front surface. One of the plurality of sensing elements includes a magnetic layer, a opposing magnetic layer, and a nonmagnetic intermediate layer. The structure body is arranged with the first sensor unit along the arrangement direction of the plurality of sensing elements. The structure body includes a structure body layer, a opposing structure body layer, and a intermediate structure body layer. The structure body layer has at least one of a floating potential with respect to the opposing structure body layer or same potential as a potential of the opposing structure body layer.

This application is based upon and claims the benefit of priority from Japanese Patent Application No. 2016-029093, filed on Feb. 18, 2016; the entire contents of which are incorporated herein by reference.

### FIELD

Embodiments of the invention relate generally to a pressure sensor, a pressure sensor system, a microphone, a blood pressure sensor, and a touch panel.

### BACKGROUND

A pressure sensor that uses a magnetic layer has been proposed. For example, the pressure sensor is applied to a microphone, a blood pressure sensor, a touch panel, etc. It is desirable to increase the sensitivity of the pressure sensor.

### BRIEF DESCRIPTION OF THE DRAWINGS

FIG. 1A to FIG. 1D are schematic views illustrating a pressure sensor according to a first embodiment;

FIG. 2A and FIG. 2B are graphs illustrating characteristics of the pressure sensor;

FIG. 3 is a schematic cross-sectional view illustrating one portion of the pressure sensor according to the first embodiment;

FIG. 4A and FIG. 4B are schematic views illustrating one portion of a pressure sensor according to a second embodiment;

FIG. 5A to FIG. 5C are schematic views illustrating the pressure sensor according to the embodiment;

FIG. 6A to FIG. 6C are schematic perspective views illustrating the pressure sensor according to the embodiment;

FIG. 7 is a schematic cross-sectional view illustrating a pressure sensor system according to a third embodiment;

FIG. 8A and FIG. 8B are schematic views illustrating electrode portions of the pressure sensor according to the embodiment;

FIG. 9A and FIG. 9B are schematic views illustrating the electrode portions of the pressure sensor according to the embodiment;

FIG. 10 is a graph illustrating characteristics of the pressure sensors;

FIG. 11 is a schematic view illustrating a microphone according to a fourth embodiment;

FIG. 12 is a schematic cross-sectional view illustrating another microphone according to the fourth embodiment;

FIG. 13A and FIG. 13B are schematic views illustrating a blood pressure sensor according to a fifth embodiment; and

FIG. 14 is a schematic view illustrating a touch panel according to a sixth embodiment.

### DETAILED DESCRIPTION

According to one embodiment, a pressure sensor includes a film portion, a first sensor unit, and a first structure body. The film portion has a front surface. The film portion is deformable. The first sensor unit is separated from a first portion of an outer edge of the front surface and fixed to one portion of the front surface. The first sensor unit includes a plurality of first sensing elements arranged along the front surface. One of the plurality of first sensing elements includes a first magnetic layer, a first opposing magnetic layer, and a first nonmagnetic intermediate layer provided between the first magnetic layer and the first opposing magnetic layer. The first structure body is arranged with the first sensor unit along the arrangement direction of the plurality of first sensing elements. The first structure body includes a first structure body layer, a first opposing structure body layer, and a first intermediate structure body layer provided between the first structure body layer and the first opposing structure body layer. The first structure body layer has at least one of a floating potential with respect to the first opposing structure body layer or same potential as a potential of the first opposing structure body layer.

According to one embodiment, a pressure sensor includes a film portion, a first sensor unit, and a first structure body. The film portion has a front surface. The film portion is deformable. The first sensor unit is separated from a first portion of an outer edge of the front surface and fixed to one portion of the front surface. The first sensor unit includes a plurality of first sensing elements arranged along the front surface. One of the plurality of first sensing elements includes a first magnetic layer, a first opposing magnetic layer, and a first nonmagnetic intermediate layer provided between the first magnetic layer and the first opposing magnetic layer. The first structure body is arranged with the first sensor unit along the arrangement direction of the plurality of first sensing elements. The first structure body includes a first structure body layer, a first opposing structure body layer, and a first intermediate structure body layer provided between the first structure body layer and the first opposing structure body layer. The first structure body layer has at least one of a floating potential with respect to the first opposing structure body layer or same potential as a potential of the first opposing structure body layer. The first sensor unit includes: a first electrode electrically connected to the first magnetic layer; and a second electrode electrically connected to the first opposing magnetic layer. The first magnetic layer is provided between the first electrode and the second electrode. The first opposing magnetic layer is provided between the first magnetic layer and the second electrode. the film portion including: a first region overlapping the first electrode, the first magnetic layer and the second electrode; a second region not overlapping the first electrode, the first magnetic layer and the second electrode; a third region overlapping the first electrode, the first magnetic layer and the second electrode; and a fourth region not overlapping the first electrode, the first magnetic layer and the second electrode. The second region is located between the first region and the third region. The third region is located between the second region and the fourth region.

According to one embodiment, a pressure sensor system includes a film portion, a first sensor unit, a first structure body and a controller. The film portion has a front surface. The film portion is deformable. The first sensor unit is separated from a first portion of an outer edge of the front surface and fixed to one portion of the front surface. The first sensor unit includes a plurality of first sensing elements arranged along the front surface. One of the plurality of first sensing elements includes a first magnetic layer, a first opposing magnetic layer, and a first nonmagnetic intermediate layer provided between the first magnetic layer and the first opposing magnetic layer. The first structure body is arranged with the first sensor unit along the arrangement direction of the plurality of first sensing elements. The first structure body includes a first structure body layer, a first opposing structure body layer, and a first intermediate structure body layer provided between the first structure body layer and the first opposing structure body layer. The controller is connected with the first sensor unit and the first structure body. The controller is configured to supply a current to the first sensor unit. The controller is configured to electrically connect the first structure body layer with the first opposing structure body layer, or to make an electrical potential of the first structure body layer floating with respect to an electrical potential of the first opposing structure body.

According to one embodiment, a microphone includes the pressure sensor described above.

According to one embodiment, a blood pressure sensor includes the pressure sensor described above.

According to one embodiment, a touch panel includes the pressure sensor described above.

Various embodiments will be described hereinafter with reference to the accompanying drawings.

The drawings are schematic and conceptual; and the relationships between the thickness and width of portions, the proportions of sizes among portions, etc., are not necessarily the same as the actual values thereof. Further, the dimensions and proportions may be illustrated differently among drawings, even for identical portions.

In the specification and drawings, components similar to those described or illustrated in a drawing thereinabove are marked with like reference numerals, and a detailed description is omitted as appropriate.

### First Embodiment

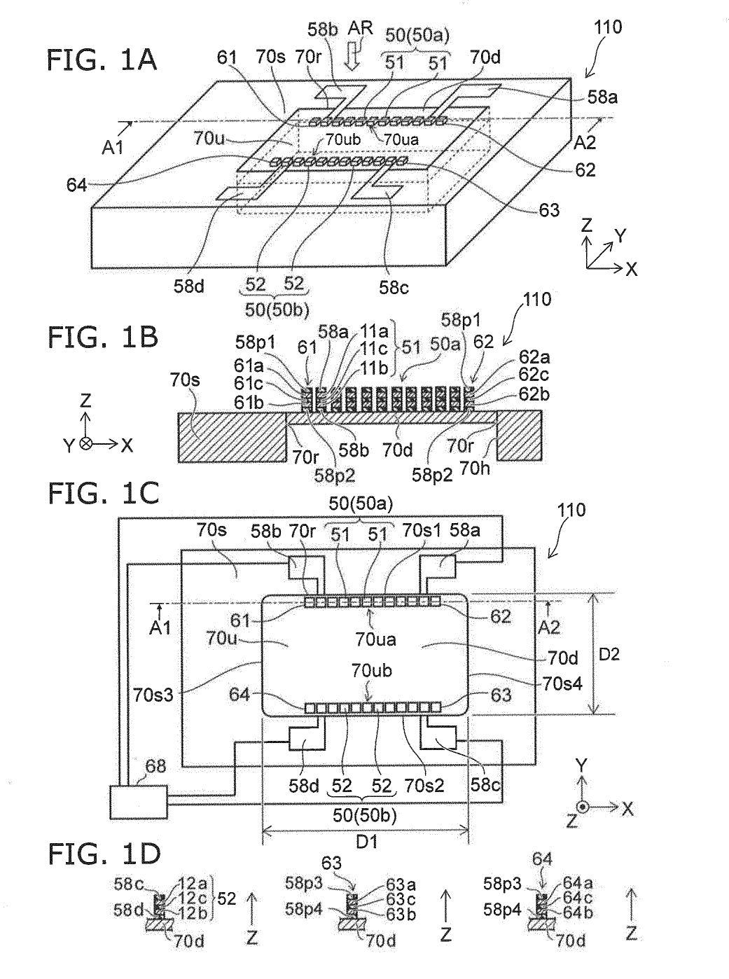

FIG. 1A to FIG. 1D are schematic views illustrating a pressure sensor according to a first embodiment.

FIG. 1A is a perspective view. FIG. 1B is a line A1-A2 cross-sectional view of FIG. 1A. FIG. 1C is a plan view as viewed along arrow AR of FIG. 1A. FIG. 1D is a cross-sectional view illustrating one portion of the pressure sensor.

As shown in FIG. 1A, the pressure sensor 110 according to the embodiment includes a film portion 70d, a first sensor unit 50a, a second sensor unit 50b, and first to fourth structure bodies 61 to 64.

The film portion 70d is deformable. The film portion 70d has a front surface 70u. The first sensor unit 50a is fixed to one portion 70ua of the front surface 70u. The first sensor unit 50a is not provided on the entire surface of the front surface 70u. One portion 70ua of the front surface 70u is separated from a first portion of an outer edge 70r of the front surface 70u. The first portion is, for example, one of first to fourth sides 70s1 to 70s4 described below. The first sensor unit 50a includes multiple first sensing elements 51. The second sensor unit 50b is fixed to another one portion 70ub of the front surface 70u. The second sensor unit 50b includes multiple second sensing elements 52. Thus, the multiple sensing elements 50 are provided. The multiple first sensing elements 51 are one portion of the multiple sensing elements 50. The multiple second sensing elements 52 are one portion of the multiple sensing elements 50.

The direction from the film portion 70d toward the first sensing elements 51 is taken as a Z-axis direction. One direction perpendicular to the Z-axis direction is taken as an X-axis direction. A direction perpendicular to the Z-axis direction and the X-axis direction is taken as a Y-axis direction.

In the example, the multiple first sensing elements 51 are arranged along the X-axis direction. The multiple second sensing elements 52 are arranged along the X-axis direction. For example, the second sensing elements 52 are arranged in the Y-axis direction with the first sensing elements. For example, at least one portion of the multiple first sensing elements 51 is connected in series to each other. For example, at least one portion of the multiple second sensing elements 52 is connected in series to each other. In the embodiment, the number of the first sensing elements 51 is arbitrary. The number of the second sensing elements 52 is arbitrary.

The film portion 70d is held by a holder 70s. The holder 70s holds the outer edge 70r. For example, a substrate that is used to form the film portion 70d and the holder 70s is provided. The substrate is, for example, a silicon substrate. A hollow 70h is provided in the substrate by removing one portion of the substrate. The thin portion of the substrate is used to form the film portion 70d. The thick portion of the substrate is used to form the holder 70s.

As shown in FIG. 1B, the first sensing element 51 includes a first magnetic layer 11a, a first opposing magnetic layer 11b, and a first nonmagnetic intermediate layer 11c. The first nonmagnetic intermediate layer 11c is provided between the first magnetic layer 11a and the first opposing magnetic layer 11b. The first opposing magnetic layer 11b is separated from the first magnetic layer 11a substantially along the Z-axis direction. In the example, the first opposing magnetic layer 11b is provided between the first magnetic layer 11a and the film portion 70d. In the embodiment, the first magnetic layer 11a may be disposed between the first opposing magnetic layer 11b and the film portion 70d.

As shown in FIG. 1D, the second sensing element 52 includes a second magnetic layer 12a, a second opposing magnetic layer 12b, and a second nonmagnetic intermediate layer 12c. The second nonmagnetic intermediate layer 12c is provided between the second magnetic layer 12a and the second opposing magnetic layer 12b. The second opposing magnetic layer 12b is separated from the second magnetic layer 12a substantially along the Z-axis direction. In the example, the second opposing magnetic layer 12b is provided between the second magnetic layer 12a and the film portion 70d. In the embodiment, the second magnetic layer 12a may be disposed between the second opposing magnetic layer 12b and the film portion 70d.

A first structure body 61 is arranged with the first sensor unit 50a in the X-axis direction. The first structure body 61 is provided between a third side 70s3 (described below) and one end of the first sensor unit 50a. The first structure body 61 is not electrically connected to the first sensor unit 50a.

The first structure body 61 includes a first structure body layer 61a, a first opposing structure body layer 61b, and a first intermediate structure body layer 61c. The first intermediate structure body layer 61c is provided between the first structure body layer 61a and the first opposing structure body layer 61b. The first opposing structure body layer 61b is separated from the first structure body layer 61a substantially along the Z-axis direction. In the example, the first opposing structure body layer 61b is provided between the first structure body layer 61a and the film portion 70d. In the embodiment, the first structure body layer 61a may be disposed between the first opposing structure body layer 61b and the film portion 70d.

The first structure body layer 61a has at least one of a floating potential with respect to the first opposing structure body layer 61b or the same potential as the potential of the first opposing structure body layer 61b.

The first structure body layer 61a includes, for example, the same material as a material included in the first magnetic layer 11a. The first opposing structure body layer 61b includes, for example, the same material as a material included in the first opposing magnetic layer 11b. The first intermediate structure body layer 61c includes, for example, the same material as a material included in the first nonmagnetic intermediate layer 11c. For example, although the structure of the first structure body 61 is substantially the same as the structure of the first sensing element 51, the first structure body 61 does not function as the sensing element 50.

For example, the first structure body layer 61a is formed from a magnetic film that is used to form the first magnetic layer 11a. For example, the first opposing structure body layer 61b is formed from a magnetic film that is used to form the first opposing magnetic layer 11b. For example, the first intermediate structure body layer 61c is formed from a nonmagnetic film that is used to form the first nonmagnetic intermediate layer 11c.

The sensitivity of the pressure sensor can be increased by increasing the number of the sensing elements 50 of the sensor unit. For example, in the case where the multiple sensing elements 50 are connected in series, the signal voltage is N times and the noise is N^{1/2} times according to the number N of the sensing elements 50. The SN ratio SNR is improved by increasing the number.

On the other hand, it was found that the amount of strain of the sensing element 50 positioned at the end of the sensor unit is small compared to the amount of strain of the sensing element 50 positioned at the center of the sensor unit. For example, it was found that the SN ratio SNR decreases when the sensing element 50 positioned at the end of the sensor unit is electrically connected to the other sensing elements 50 of the sensor unit.

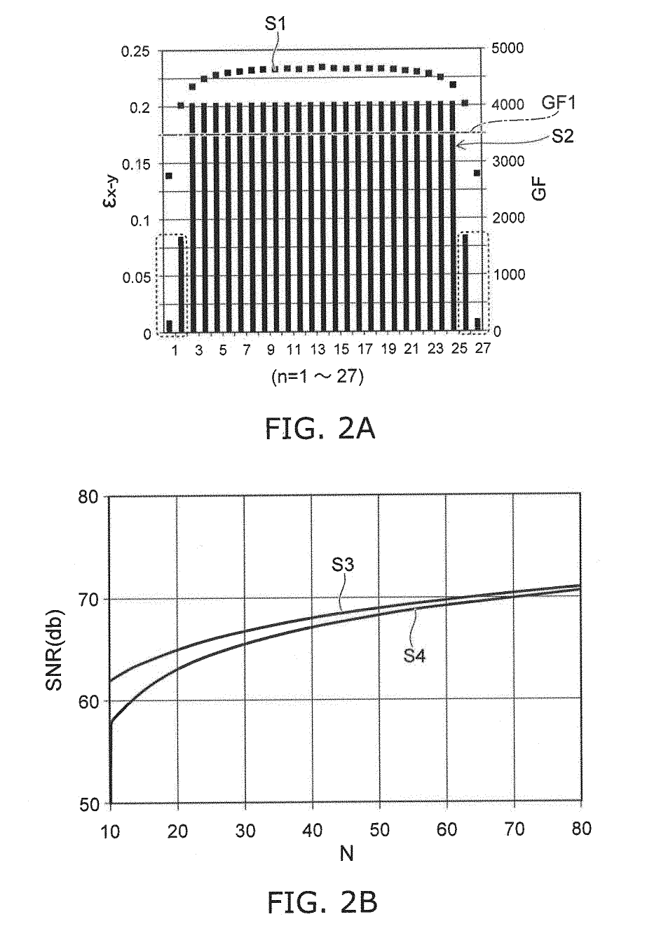

FIG. 2A and FIG. 2B are graphs illustrating characteristics of the pressure sensor.

In FIG. 2A, the horizontal axis shows the position of each of the sensing elements 50. The vertical axis shows an anisotropic strain ε_{x-y} or a gauge factor GF. The gauge factor GF is the change amount (dR/R) of the electrical resistance per unit strain (dε). For example, the sensitivity is high when the gauge factor GF is high.

In the example, the multiple sensing elements 50 are arranged in series. The number of the multiple sensing elements 50 is 27. In the example, the anisotropic strain ε_{x-y} and the gauge factor GF are shown for the sensing element 50 at each of first to twenty-seventh positions. A characteristic S1 shows the anisotropic strain ε_{x-y}. The anisotropic strain ε_{x-y} is small for the sensing elements 50 at the two ends compared to the sensing elements 50 at the center. A characteristic S2 shows the gauge factor GF of each of the sensing elements 50. The gauge factor GF is small for the sensing elements 50 at the two ends compared to the sensing elements 50 at the center. For example, the gauge factors GF of the sensing elements 50 at the first, second, twenty-sixth, and twenty-seventh positions are lower than a reference value GF1. The desired sensitivity (e.g., 70 dB) is obtained at and above the reference value GF1. The SN ratio SNR decreases when the sensing elements 50 at the two ends are connected to the sensing elements 50 at the center.

In the embodiment, for example, the first structure body 61 is disposed instead of the sensing element 50 of the first position. For example, the second structure body 62 is disposed instead of the sensing element 50 of the twenty-seventh position. The first structure body 61 and the second structure body 62 are not electrically connected to the sensing elements 50 of the second to twenty-sixth positions. In the embodiment, the decrease of the SN ratio SNR can be suppressed. Thereby, the sensitivity can be increased.

In FIG. 2B, the horizontal axis shows the number N of the multiple sensing elements 50. The vertical axis shows the SN ratio SNR (dB). A characteristic S3 shows the SN ratio SNR when the first structure body 61 is disposed at one end of the multiple sensing elements 50, and the second structure body 62 is disposed at the other end. The first structure body 61 and the second structure body 62 are not included in the number N. A characteristic S4 shows the SN ratio SNR when only the multiple sensing elements 50 are disposed, and the first structure body 61 and the second structure body 62 are not disposed. In the example, the SN ratio SNR of the characteristic S3 is relatively higher than the SN ratio SNR of the characteristic S4 when the number N is not less than 10 and not more than 80. In other words, by disposing the first structure body 61 and the second structure body 62, the decrease of the SN ratio SNR can be suppressed.

In the example as shown in FIG. 1C, the film portion 70d has the outer edge 70r. The outer edge 70r is substantially a polygon (a quadrilateral, and specifically a rectangle). The outer edge 70r includes the first side 70s1, the second side 70s2, the third side 70s3, and the fourth side 70s4.

Various configurations are applicable to the film portion 70d (the outer edge 70r). The film portion 70d (the outer edge 70r) may have, for example, a substantially perfect circle configuration, may have a flattened circular configuration (including an elliptical configuration), may have a substantially square configuration, or may have a rectangular configuration. For example, in the case where the film portion 70d (the outer edge 70r) has a substantially square configuration or a substantially rectangular configuration, the portions at the four corners (the corner portions) may have curved configurations.

The first side 70s1 extends in a first direction (in the example, the X-axis direction). The second side 70s2 is separated from the first side 70s1 in a second direction. The second direction crosses the first direction. In the example, the second direction is the Y-axis direction. The second side 70s2 extends in the first direction (the X-axis direction). The third side 70s3 extends in the second direction (the Y-axis direction). The fourth side 70s4 extends in the second direction (the Y-axis direction) and is separated from the third side 70s3 in the first direction (the X-axis direction).

In the example, a distance D1 along the first direction between the first side 70s1 and the second side 70s2 is longer than a distance D2 along the second direction between the third side 70s3 and the fourth side 70s4. The film portion 70d is substantially a rectangle; and the first side 70s1 and the second side 70s2 are the long sides. The third side 70s3 and the fourth side 70s4 are the short sides.

In the embodiment as illustrated in FIG. 1C, a curved portion may be provided in the outer edge 70r between the sides. For example, the corner portions of the film portion 70d (the outer edge 70r) have curved configurations. Thereby, for example, the strength of the film portion 70d is increased.

A large strain (anisotropic strain) occurs at the vicinity of the outer edge 70r of the film portion 70d when stress is applied to the film portion 70d. By disposing the sensing elements 50 at the vicinity of the outer edge 70r of the film portion 70d, a large strain is applied to the sensing elements 50; and a high sensitivity is obtained. In particular, in the case where one length of the film portion 70d is longer than the length in the other direction (i.e., in the case where the configuration is anisotropic), a particularly large strain occurs in the portion along the major axis inside the outer edge 70r. Therefore, by disposing the sensing elements 50 in the portion along the long side of the outer edge 70r, a particularly high sensitivity is obtained.

In the example, the multiple first sensing elements 51 are arranged along the first side 70s1. The multiple second sensing elements 52 are arranged along the second side 70s2. In the case where one length of the film portion 70d is longer than the other length of the film portion 70d (in the case where the configuration is anisotropic), the region where the anisotropic strain occurs at the end portion vicinity on the minor axis side of the film portion 70d is wide compared to the case where the film portion 70d has an isotropic configuration.

Anisotropic strain having a larger absolute value occurs in a wider region for the end portion on the minor axis side of the film portion 70d having the anisotropic configuration than for the end portion of the film portion 70d having the isotropic configuration. More sensing elements 50 can be disposed in the film portion 70d having the anisotropic configuration than in the film portion 70d having the isotropic configuration. The sensing elements 50 that are disposed are sensing elements 50 in which a similar change of the electrical resistance (e.g., having the same polarity) occurs according to the pressure. Thereby, a highly-sensitive pressure sensor can be provided.

By connecting the multiple sensing elements 50 in series, the SN ratio can be improved. In the embodiment, the multiple sensing elements 50 can be disposed in which electrical signals of the same polarity are obtained when the pressure is applied. Thereby, the SN ratio improves.

The second structure body 62 may be provided in the embodiment. The second structure body 62 is arranged with the first sensor unit 50a in the X-axis direction. The second structure body 62 is provided between the fourth side 70s4 and the other end of the first sensor unit 50a. The second structure body 62 is not electrically connected to the first sensor unit 50a. The second structure body 62 includes a second structure body layer 62a, a second opposing structure body layer 62b, and a second intermediate structure body layer 62c. The second intermediate structure body layer 62c is provided between the second structure body layer 62a and the second opposing structure body layer 62b. The second opposing structure body layer 62b is separated from the second structure body layer 62a substantially along the Z-axis direction. In the example, the second opposing structure body layer 62b is provided between the second structure body layer 62a and the film portion 70d. In the embodiment, the second structure body layer 62a may be disposed between the second opposing structure body layer 62b and the film portion 70d. The second structure body layer 62a has at least one of a floating potential with respect to the second opposing structure body layer 62b or the same potential as the potential of the second opposing structure body layer 62b.

The second structure body layer 62a includes, for example, the same material as a material included in the first magnetic layer 11a. The second opposing structure body layer 62b includes, for example, the same material as a material included in the first opposing magnetic layer 11b. The second intermediate structure body layer 62c includes, for example, the same material as a material included in the second nonmagnetic intermediate layer 12c. For example, although the structure of the second structure body 62 is substantially the same as the structure of the first sensing element 51, the second structure body 62 does not function as the sensing element 50.

The third structure body 63 may be provided. The third structure body 63 is arranged with the second sensor unit 50b in the X-axis direction. The third structure body 63 is provided between the fourth side 70s4 and one end of the second sensor unit 50b. The third structure body 63 is not electrically connected to the second sensor unit 50b. The third structure body 63 includes a third structure body layer 63a, a third opposing structure body layer 63b, and a third intermediate structure body layer 63c. The third intermediate structure body layer 63c is provided between the third structure body layer 63a and the third opposing structure body layer 63b. The third opposing structure body layer 63b is separated from the third structure body layer 63a substantially along the Z-axis direction. In the example, the third opposing structure body layer 63b is provided between the third structure body layer 63a and the film portion 70d. In the embodiment, the third structure body layer 63a may be disposed between the third opposing structure body layer 63b and the film portion 70d. The third structure body layer 63a has at least one of a floating potential with respect to the third opposing structure body layer 63b or the same potential as the potential of the third opposing structure body layer 63b.

The third structure body layer 63a includes, for example, the same material as a material included in the second magnetic layer 12a. The third opposing structure body layer 63b includes, for example, the same material as a material included in the second opposing magnetic layer 12b. The third intermediate structure body layer 63c includes, for example, the same material as a material included in the second nonmagnetic intermediate layer 12c. For example, although the structure of the third structure body 63 is substantially the same as the structure of the second sensing element 52, the third structure body 63 does not function as the sensing element 50.

The fourth structure body 64 may be provided. The fourth structure body 64 is arranged with the second sensor unit 50b in the X-axis direction. The fourth structure body 64 is provided between the third side 70s3 and the other end of the second sensor unit 50b. The fourth structure body 64 is not electrically connected to the second sensor unit 50b. The fourth structure body 64 includes a fourth structure body layer 64a, a fourth opposing structure body layer 64b, and a fourth intermediate structure body layer 64c. The fourth intermediate structure body layer 64c is provided between the fourth structure body layer 64a and the fourth opposing structure body layer 64b. The fourth opposing structure body layer 64b is separated from the fourth structure body layer 64a substantially along the Z-axis direction. In the example, the fourth opposing structure body layer 64b is provided between the fourth structure body layer 64a and the film portion 70d. In the embodiment, the fourth structure body layer 64a may be disposed between the fourth opposing structure body layer 64b and the film portion 70d. The fourth structure body layer 64a has at least one of a floating potential with respect to the fourth opposing structure body layer 64b or the same potential as the potential of the fourth opposing structure body layer 64b.

The fourth structure body layer 64a includes, for example, the same material as a material included in the second magnetic layer 12a. The fourth opposing structure body layer 64b includes, for example, the same material as a material included in the second opposing magnetic layer 12b. The fourth intermediate structure body layer 64c includes, for example, the same material as the material included in the second nonmagnetic intermediate layer 12c. For example, although the structure of the fourth structure body 64 is substantially the same as the structure of the second sensing element 52, the fourth structure body 64 does not function as the sensing element 50.

The magnetization of the first magnetic layer 11a changes according to the deformation of the film portion 70d. The magnetization of the second magnetic layer 12a changes according to the deformation of the film portion 70d. The first magnetic layer 11a is, for example, a free magnetic layer. The second magnetic layer 12a is, for example, a free magnetic layer.

For example, the magnetization of the first opposing magnetic layer 11b does not change easily compared to the magnetization of the first magnetic layer 11a. The first opposing magnetic layer 11b is, for example, a fixed magnetic layer. For example, the magnetization of the second opposing magnetic layer 12b does not change easily compared to the magnetization of the second magnetic layer 12a. The second opposing magnetic layer 12b is, for example, a fixed magnetic layer.

For example, pressure (the pressure to be sensed) is applied to the film portion 70d. Thereby, strain occurs in the magnetic layers of the sensing elements 50. The strain is, for example, anisotropic strain. Due to the strain, the magnetization of the first magnetic layer 11a and the magnetization of the second magnetic layer 12a each change. For example, the changes are based on the inverse magnetostrictive effect. Thereby, the angle between the direction of the magnetization of the first magnetic layer 11a and the direction of the magnetization of the first opposing magnetic layer 11b changes. Thereby, the resistance between the first magnetic layer 11a and the first opposing magnetic layer 11b changes. On the other hand, the angle between the direction of the magnetization of the second magnetic layer 12a and the direction of the magnetization of the second opposing magnetic layer 12b changes. Thereby, the resistance between the second magnetic layer 12a and the second opposing magnetic layer 12b changes. For example, the changes of the resistances are based on the magnetoresistance effect (the MR effect).

In other words, the resistance between the first magnetic layer 11a and the first opposing magnetic layer 11b changes according to the deformation of the film portion 70d. The angle between the direction of the magnetization of the second magnetic layer 12a and the direction of the magnetization of the second opposing magnetic layer 12b changes. By sensing the changes of the resistances, the pressure that is applied to the film portion 70d is sensed. In other words, the pressure that is to be sensed is sensed.

For example, the change of the resistance is sensed by causing a current to flow in the sensing elements 50.

As illustrated in FIG. 1B, the first sensor unit 50a further includes, for example, a first electrode 58a and a second electrode 58b. For example, the first magnetic layer 11a, the first opposing magnetic layer 11b, and the first nonmagnetic intermediate layer 11c are disposed between the first electrode 58a and the second electrode 58b. The resistance of the first sensing element 51 is sensed by applying a voltage between the first electrode 58a and the second electrode 58b.

As illustrated in FIG. 1D, the second sensor unit 50b further includes, for example, a third electrode 58c and a fourth electrode 58d. For example, the second magnetic layer 12a, the second opposing magnetic layer 12b, and the second nonmagnetic intermediate layer 12c are disposed between the third electrode 58c and the fourth electrode 58d. The resistance of the second sensing element 52 is sensed by applying a voltage between the third electrode 58c and the fourth electrode 58d.

In the embodiment, the first structure body 61 further includes a first conductive layer 58p1 and a second conductive layer 58p2. The first conductive layer 58p1 is electrically connected to the first structure body layer 61a. The second conductive layer 58p2 is electrically connected to the first opposing structure body layer 61b. The first structure body layer 61a is provided between the first conductive layer 58p1 and the second conductive layer 58p2. The first opposing structure body layer 61b is provided between the first structure body layer 61a and the second conductive layer 58p2. A voltage is not applied to the first structure body 61 because the first structure body 61 is not electrically connected to the first sensing element 51.

The first conductive layer 58p1 is further electrically connected to the second structure body layer 62a. The second conductive layer 58p2 is electrically connected to the second opposing structure body layer 62b. The second structure body layer 62a is provided between the first conductive layer 58p1 and the second conductive layer 58p2. The second opposing structure body layer 62b is provided between the second structure body layer 62a and the second conductive layer 58p2. A voltage is not applied to the second structure body 62 because the second structure body 62 is not electrically connected to the first sensing element 51.

The third structure body 63 further includes a third conductive layer 58p3 and a fourth conductive layer 58p4. The third conductive layer 58p3 is electrically connected to the third structure body layer 63a. The fourth conductive layer 58p4 is electrically connected to the third opposing structure body layer 63b. The third structure body layer 63a is provided between the third conductive layer 58p3 and the fourth conductive layer 58p4. The third opposing structure body layer 63b is provided between the third structure body layer 63a and the fourth conductive layer 58p4. A voltage is not applied to the third structure body 63 because the third structure body 63 is not electrically connected to the second sensing element 52.

The third conductive layer 58p3 is further electrically connected to the fourth structure body layer 64a. The fourth conductive layer 58p4 is electrically connected to the fourth opposing structure body layer 64b. The fourth structure body layer 64a is provided between the third conductive layer 58p3 and the fourth conductive layer 58p4. The fourth opposing structure body layer 64b is provided between the fourth structure body layer 64a and the fourth conductive layer 58p4. A voltage is not applied to the fourth structure body 64 because the fourth structure body 64 is not electrically connected to the second sensing element 52.

The magnetic layers (the first magnetic layer 11a and the second magnetic layer 12a) include, for example, at least one of Fe, Co, or Ni. The opposing magnetic layers (the first opposing magnetic layer 11b and the second opposing magnetic layer 12b) include, for example, at least one of Fe, Co, or Ni. The nonmagnetic intermediate layers (the first nonmagnetic intermediate layer 11c and the second nonmagnetic intermediate layer 12c) may include a metal or an insulator. In the case of a metal, for example, Cu, Au, Ag, or the like is used. In the case of an insulator, for example, magnesium oxide, aluminum oxide, titanium oxide, zinc oxide, or the like is used.

An insulating layer (not illustrated) is provided between the first electrode 58a and the film portion 70d. For example, the insulating layer is provided also between the first electrode 58a and the second electrode 58b. For example, the insulating layer is provided also between the third electrode 58c and the fourth electrode 58d. Electrical insulation between the electrodes is obtained due to the insulating layer.

As shown in FIG. 1C, a controller 68 (e.g., a processing circuit) may be further provided. The controller 68 is electrically connected to the first sensing element 51 and the second sensing element 52. For example, the controller 68 is electrically connected to the first electrode 58a, the second electrode 58b, the third electrode 58c, and the fourth electrode 58d. The controller 68 outputs a signal corresponding to the signal obtained from the first sensing element 51 (the signal generated by the first sensing element 51). The controller 68 outputs a signal corresponding to the signal obtained from the second sensing element 52 (the signal generated by the second sensing element 52). The controller 68 outputs a signal corresponding to the change of the resistance occurring in the sensing elements 50. The signals obtained by the controller 68 correspond to the pressure to be sensed.

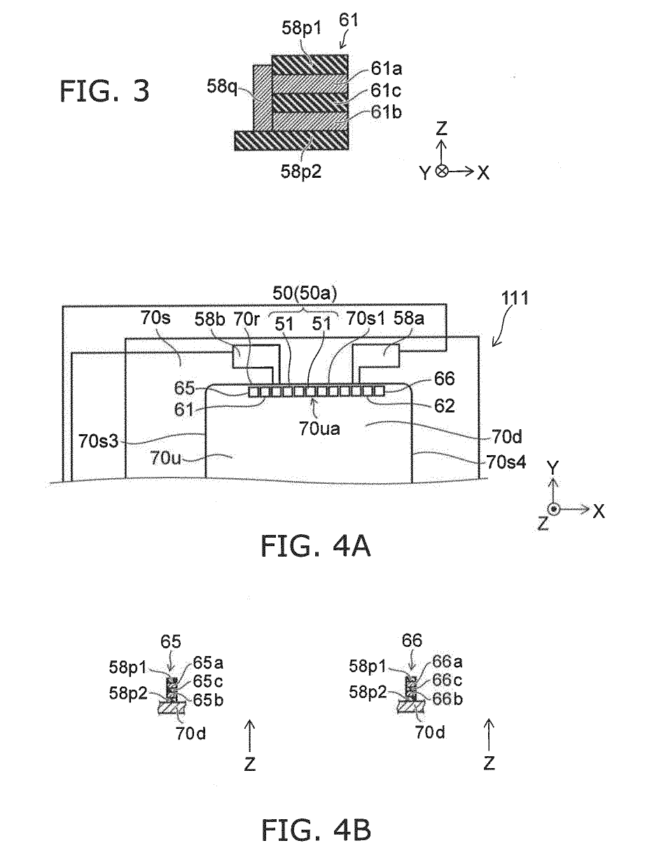

FIG. 3 is a schematic cross-sectional view illustrating one portion of the pressure sensor according to the first embodiment.

As shown in FIG. 3, the first structure body 61 further includes the first conductive layer 58p1, the second conductive layer 58p2, and an interconnect layer 58q. The first structure body layer 61a is provided between the first conductive layer 58p1 and the second conductive layer 58p2. The length along the X-axis direction of the second conductive layer 58p2 on the lower side is longer than the length along the X-axis direction of the first conductive layer 58p1 on the upper side. The interconnect layer 58q electrically connects the first conductive layer 58p1 and the second conductive layer 58p2. The first structure body layer 61a has the same potential as the potential of the first opposing structure body layer 61b. This is similar for the second to fourth structure bodies 62 to 64 as well.

According to the embodiment, the structure body that is not electrically connected to the end of the sensor unit is disposed. The structure body does not function as the sensing element 50. Therefore, the decrease of the SNR can be suppressed. Thereby, the sensitivity can be increased.

### Second Embodiment

FIG. 4A and FIG. 4B are schematic views illustrating one portion of a pressure sensor according to a second embodiment.

FIG. 4A is a plan view illustrating one portion of the pressure sensor.

FIG. 4B is a cross-sectional view illustrating one portion of the pressure sensor.

The pressure sensor 111 according to the embodiment further includes a fifth structure body 65 and a sixth structure body 66. The fifth structure body 65 is arranged with the first sensor unit 50a in the X-axis direction. The fifth structure body 65 is not electrically connected to the first sensor unit 50a. The fifth structure body 65 is provided between the first structure body 61 and the third side 70s3. The sixth structure body 66 is arranged with the first sensor unit 50a in the X-axis direction. The sixth structure body 66 is not electrically connected to the first sensor unit 50a. The sixth structure body 66 is provided between the second structure body 62 and the fourth side 70s4.

The fifth structure body 65 includes a fifth structure body layer 65a, a fifth opposing structure body layer 65b, and a fifth intermediate structure body layer 65c. The fifth intermediate structure body layer 65c is provided between the fifth structure body layer 65a and the fifth opposing structure body layer 65b. The fifth opposing structure body layer 65b is separated from the fifth structure body layer 65a substantially along the Z-axis direction. In the example, the fifth opposing structure body layer 65b is provided between the fifth structure body layer 65a and the film portion 70d. In the embodiment, the fifth structure body layer 65a may be disposed between the fifth opposing structure body layer 65b and the film portion 70d. The fifth structure body layer 65a has at least one of a floating potential with respect to the fifth opposing structure body layer 65b or the same potential as the potential of the fifth opposing structure body layer 65b.

The fifth structure body layer 65a includes, for example, the same material as a material included in the first magnetic layer 11a. The fifth opposing structure body layer 65b includes, for example, the same material as a material included in the first opposing magnetic layer 11b. The fifth intermediate structure body layer 65c includes, for example, the same material as a material included in the first nonmagnetic intermediate layer 11c. For example, although the structure of the fifth structure body 65 is substantially the same as the structure of the first sensing element 51, the fifth structure body 65 does not function as the sensing element 50.

The sixth structure body 66 includes a sixth structure body layer 66a, a sixth opposing structure body layer 66b, and a sixth intermediate structure body layer 66c. The sixth intermediate structure body layer 66c is provided between the sixth structure body layer 66a and the sixth opposing structure body layer 66b. The sixth opposing structure body layer 66b is separated from the sixth structure body layer 66a substantially along the Z-axis direction. In the example, the sixth opposing structure body layer 66b is provided between the sixth structure body layer 66a and the film portion 70d. In the embodiment, the sixth structure body layer 66a may be disposed between the sixth opposing structure body layer 66b and the film portion 70d. The sixth structure body layer 66a has at least one of a floating potential with respect to the sixth opposing structure body layer 66b or the same potential as the potential of the sixth opposing structure body layer 66b.

The sixth structure body layer 66a includes, for example, the same material as a material included in the first magnetic layer 11a. The sixth opposing structure body layer 66b includes, for example, the same material as a material included in the first opposing magnetic layer 11b. The sixth intermediate structure body layer 66c includes, for example, the same material as a material included in the first nonmagnetic intermediate layer 11c. For example, although the structure of the sixth structure body 66 is substantially the same as the structure of the first sensing element 51, the sixth structure body 66 does not function as the sensing element 50.

The number of structure bodies disposed at one end of the second sensor unit 50b and at the other end of the second sensor unit 50b may be 2 each,

As described in reference to FIG. 2A, the gauge factors GF are small for the two sensing elements 50 provided at the end on one side of the sensor unit. In the embodiment, these sensing elements 50 (the number being 4) are structure bodies that are not electrically connected. The structure bodies do not function as the sensing elements 50. For example, the fifth structure body 65 is disposed instead of the sensing element 50 of the first position. The first structure body 61 is disposed instead of the sensing element 50 of the second position. The second structure body 62 is disposed instead of the sensing element 50 of the twenty-sixth position. The sixth structure body 66 is disposed instead of the sensing element 50 of the twenty-seventh position. First, second, fifth, and sixth structure bodies 61, 62, 65, and 66 are not electrically connected to the sensing elements 50 of the third to twenty-fifth positions. Therefore, the decrease of the SNR can be suppressed. Thereby, the sensitivity can be increased.

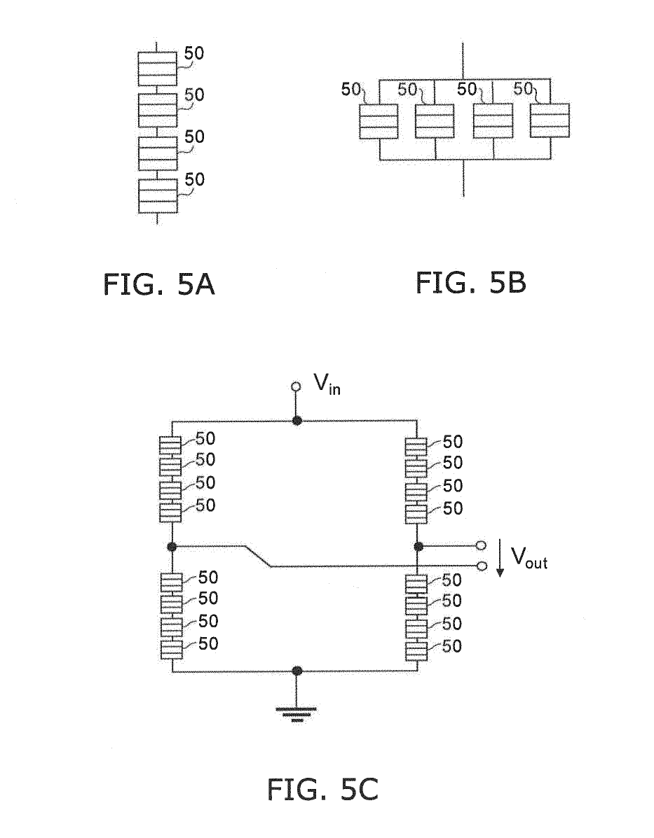

FIG. 5A to FIG. 5C are schematic views illustrating the pressure sensor according to the embodiment.

These drawings show examples of the connection states of the multiple sensing elements 50.

In FIG. 5A, the sensing elements 50 correspond to the first sensing element 51, the second sensing element 52, etc. The multiple sensing elements 50 are connected in series. The number of the multiple sensing elements 50 connected in series is N. Therefore, the electrical signal that is obtained is N times that of the case where the number of the sensing elements 50 is 1. On the other hand, the thermal noise and the Schottky noise are N^{1/2} times. In other words, SNR is N^{1/2} times. By increasing the number N of the sensing elements 50 connected in series, the SN ratio can be improved without increasing the size of the film portion 70d.

In the embodiment, by using the film portion 70d having the anisotropic configuration, the change (e.g., the polarity) of the electrical resistance according to the pressure is similar for each of the multiple sensing elements 50 disposed to be clustered at the center of gravity vicinity of the film portion 70d. Therefore, it is possible to add the signals of each of the multiple sensing elements 50.

The bias voltage that is applied to one sensing element 50 is, for example, not less than 50 millivolts (mV) and not more than 150 mV. In the case where the N sensing elements 50 are connected in series, the bias voltage is not less than 50 mV×N and not more than 150 mV×N. For example, in the case where the number N of the sensing elements 50 connected in series is 25, the bias voltage is not less than 1 V and not more than 3.75 V.

When the value of the bias voltage is 1 V or more, the design of the electronic circuit processing the electrical signals obtained from the sensing elements 50 is easy and is practically favorable. In the embodiment, the sensing elements 50 can be multiply disposed in which the electrical signals obtained have the same polarity when the pressure is applied. Therefore, these sensing elements 50 are connected in series; and the SN ratio can be improved as recited above.

For the electronic circuit that processes the electrical signals obtained from the sensing elements 50, it is undesirable for the bias voltage (the voltage across the terminals) to exceed 10 V. In the embodiment, the bias voltage and the number N of the sensing elements 50 connected in series are set to provide the appropriate voltage range.

For example, it is favorable for the voltage when the multiple sensing elements 50 are connected electrically in series to be not less than 1 V and not more than 10 V. For example, the voltage that is applied between the terminals of the multiple sensing elements 50 connected electrically in series (between the terminal of one end and the terminal of the other end) is not less than 1 V and not more than 10 V.

To generate such a voltage, in the case where the bias voltage that is applied to one sensing element 50 is 50 my, it is favorable for the number N of the sensing elements 50 connected in series to be not less than 20 and not more than 200. In the case where the bias voltage that is applied to one sensing element 50 is 150 mV, it is favorable for the number N of the sensing elements 50 connected in series to be not less than 7 and not more than 66.

As shown in FIG. 5B, at least one portion of the multiple sensing elements 50 may be electrically connected in parallel.

As shown in FIG. 5C, the multiple sensing elements 50 may be connected so that the multiple sensing elements 50 form a Wheatstone bridge circuit. Thereby, for example, temperature compensation of the sensing characteristics can be performed.

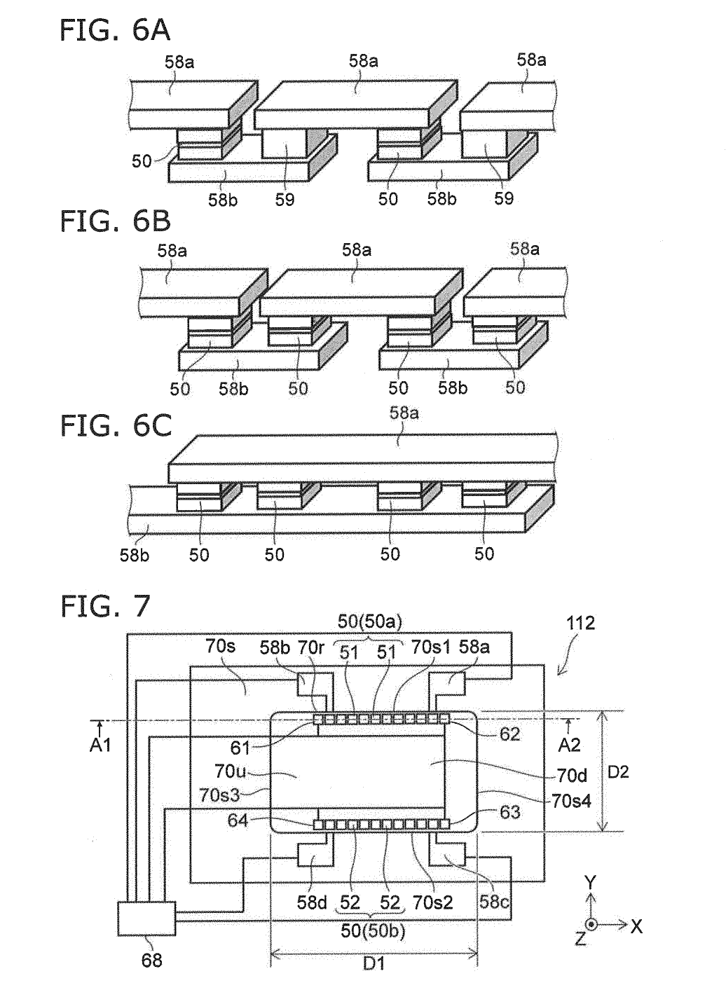

FIG. 6A to FIG. 6C are schematic perspective views illustrating the pressure sensor according to the embodiment.

These drawings show examples of the connections of the multiple sensing elements 50.

As shown in FIG. 6A, in the case where the multiple sensing elements 50 are connected electrically in series, the sensing element 50 and a via contact 59 are provided between the second electrode 58b on the lower portion side and the first electrode 58a on the upper portion side. Thereby, the conduction direction is in one direction. The current that is conducted in the multiple sensing elements 50 is downward or upward. By such a connection, the difference between the characteristics of each of the multiple sensing elements 50 can be small.

As shown in FIG. 6B, the sensing elements 50 are disposed between the second electrode 58b and the first electrode 58a without providing the via contact 59. In the example, the directions of the currents conducted in each of two mutually-adjacent sensing elements 50 are mutually reversed. The density of the arrangement of the multiple sensing elements 50 is high for such a connection.

As shown in FIG. 6C, the multiple sensing elements 50 are provided between one second electrode 58b and one first electrode 58a. The multiple sensing elements 50 are connected in parallel.

### Third Embodiment

FIG. 7 is a schematic cross-sectional view illustrating a pressure sensor system according to a third embodiment.

The pressure sensor system 112 according to the embodiment includes the film portion 70d, the first sensor unit 50a, the second sensor unit 50b, the first to fourth structure bodies 61 to 64, and the controller 68.

For example, the controller 68 is electrically connected to the first sensor unit 50a and the first structure body 61. The controller 68 electrically connects the first structure body layer 61a (referring to FIG. 1B) and the first opposing structure body layer 61b while supplying a current to the first sensor unit 50a. The controller 68 causes the potential of the first structure body layer 61a to be floating with respect to the first opposing structure body layer 61b while supplying the current to the first sensor unit 50a.

As in the embodiment, the controller 68 may control the potential of the first structure body 61. A similar control is possible for the second to sixth structure bodies 62 to 66 as well.

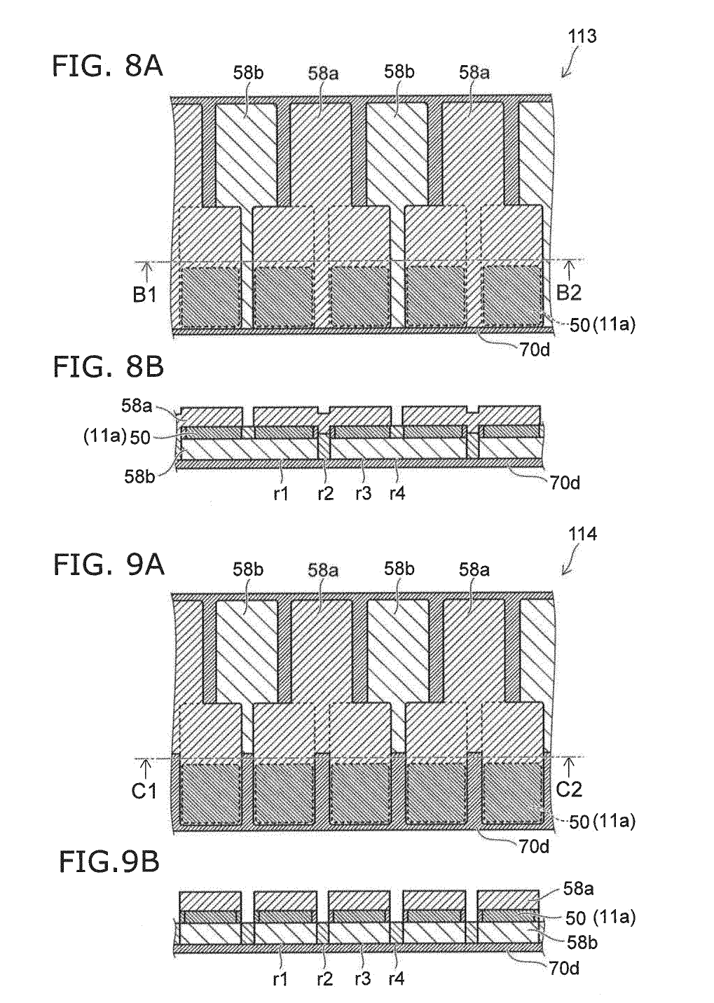

FIG. 8A and FIG. 8B are schematic views illustrating electrode portions of the pressure sensor according to the embodiment.

FIG. 8A is a schematic plan view of the electrode portions. FIG. 8B is a line B1-B2 cross-sectional view of FIG. 8A. Line B1-B2 is aligned with the outer edge 70r of the film portion 70d.

The pressure sensor 113 according to the embodiment includes the film portion 70d, the multiple sensing elements 50, the first electrode 58a, and the second electrode 58b. As shown in FIG. 8B, the film portion 70d includes a first region r1, a second region r2, a third region r3, and a fourth region r4. The first region r1 overlaps the first electrode 58a, the first magnetic layer 11a (the sensing element 50), and the second electrode 58b. The second region r2 overlaps the first electrode 58a but does not overlap the first magnetic layer 11a (the sensing element 50) or the second electrode 58b. The third region r3 overlaps the first electrode 58a, the first magnetic layer 11a (the sensing element 50), and the second electrode 58b. The fourth region r4 overlaps the second electrode 58b but does not overlap the first magnetic layer 11a (the sensing element 50) or the first electrode 58a. The second region r2 is positioned between the first region r1 and the third region r3. The third region r3 is positioned between the second region r2 and the fourth region r4. That is, on the film portion 70d, the first electrode 58a on the upper side and the second electrode 58b on the lower side are electrically connected.

FIG. 9A and FIG. 9B are schematic views illustrating the electrode portions of the pressure sensor according to the embodiment.

FIG. 9A is a schematic plan view of the electrode portions. FIG. 9B is a line C1-C2 cross-sectional view of FIG. 9A. Line C1-C2 is aligned with the outer edge 70r of the film portion 70d.

The pressure sensor 114 according to the embodiment includes the film portion 70d, the multiple sensing elements 50, the first electrode 58a, and the second electrode 58b. As shown in FIG. 9B, the film portion 70d includes the first region r1, the second region r2, the third region r3, and the fourth region r4. The first region r1 overlaps the first electrode 58a, the sensing element 50, and the second electrode 58b. The second region r2 does not overlap the first electrode 58a, the sensing element 50, or the second electrode 58b. The third region r3 overlaps the first electrode 58a, the sensing element 50, and the second electrode 58b. The fourth region r4 does not overlap the first electrode 58a, the sensing element 50, or the second electrode 58b. The second region r2 is positioned between the first region r1 and the third region r3. The third region r3 is positioned between the second region r2 and the fourth region r4. That is, on the film portion 70d, the first electrode 58a on the upper side and the second electrode 58b on the lower side are not electrically connected. In such a case, the first electrode 58a and the second electrode 58b are electrically connected inside the holder 70s.

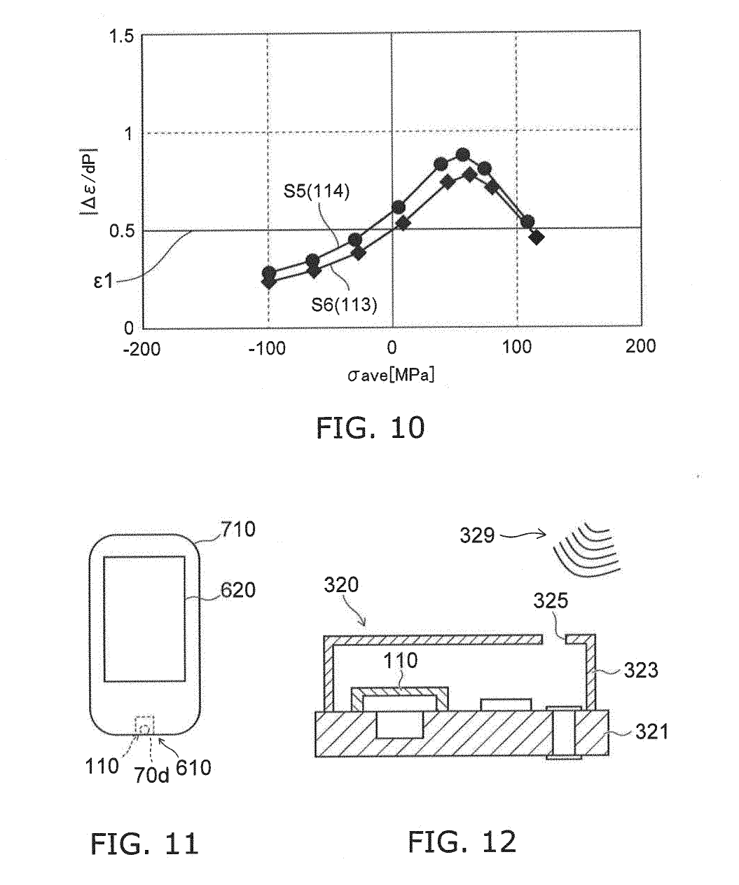

FIG. 10 is a graph illustrating characteristics of the pressure sensors.

In FIG. 10, the horizontal axis shows an element unit volumetric average stress σave (MPa). The vertical axis shows an anisotropic strain slope |Δε/dP|. The element unit volumetric average stress cave illustrates the average stress per unit volume generated in the element unit when pressure (sound pressure) is not applied. The element unit includes the first electrode 58a, the sensing element 50, and the second electrode 58b. The anisotropic strain slope |Δε/dP| illustrates the absolute value of the change amount (de) of the strain per unit stress (dP) generated in the sensing element 50.

A characteristic 55 shows the anisotropic strain slope |Δε/dP| of the pressure sensor 114. A characteristic 56 shows the anisotropic strain slope |Δε/dP| of the pressure sensor 113. In the example, the anisotropic strain slope |Δε/dP| has a peak when the element unit volumetric average stress cave is in the vicinity of +60 MPa. For example, a reference value ε1 is taken to be 0.5. The desired characteristics are obtained when the anisotropic strain slope |Δε/dP| is not less than the reference value ε1.

For example, for the element unit volumetric average stress σave at the vicinity of +60 MPa, the anisotropic strain slope |Δε/dP| (the characteristic S5) of the pressure sensor 114 is larger than the anisotropic strain slope |Δε/dP| (the characteristic 56) of the pressure sensor 113. That is, compared to the pressure sensor 113, a larger amount of strain can be obtained for the pressure sensor 114. It is considered that this is caused by the difference between the electrode structure of the pressure sensor 114 and the electrode structure of the pressure sensor 113.

In the case of the pressure sensor 113, strain of the reverse orientations occurs due to the first electrode 58a and the second electrode 58b in the sensing element 50; and the amount of strain decreases. Conversely, in the case of the pressure sensor 114, strain of the same orientation occurs due to the first electrode 58a and the second electrode 58b in the sensing element 50; and the amount of strain does not decrease. Therefore, it is considered that the amount of strain of the pressure sensor 114 is larger than the amount of strain of the pressure sensor 113. Therefore, the electrode structure of the pressure sensor 114 is more desirable than the electrode structure of the pressure sensor 113.

### Fourth Embodiment

FIG. 11 is a schematic view illustrating a microphone according to a fourth embodiment.

As shown in FIG. 11, a microphone 610 according to the embodiment includes any pressure sensor according to the embodiments or a pressure sensor according to a modification of the embodiments recited above. In the example, the pressure sensor 110 is used as the pressure sensor.

For example, the microphone 610 is provided in a personal digital assistant 710. For example, the film portion 70d of the pressure sensor 110 is substantially parallel to the surface in which a display unit 620 of the personal digital assistant 710 is provided. The disposition of the film portion 70d is arbitrary. According to the embodiment, a microphone in which the dynamic range can be enlarged can be provided. For example, the microphone 610 according to the embodiment may be provided in an IC recorder, a pin microphone, etc.

FIG. 12 is a schematic cross-sectional view illustrating another microphone according to the fourth embodiment.

A microphone 320 (an acoustic microphone) according to the embodiment includes a printed circuit board 321, a cover 323, and a pressure sensor. Any pressure sensor according to the embodiments or a modification of the embodiments is used as the pressure sensor. In the example, the pressure sensor 110 is used as the pressure sensor. The printed circuit board 321 includes, for example, a circuit such as an amplifier, etc. An acoustic hole 325 is provided in the cover 323. Sound 329 passes through the acoustic hole 325 and enters the interior of the cover 323. The microphone 320 responds to the sound pressure. A highly-sensitive microphone 320 is obtained by using the highly-sensitive pressure sensor 110. For example, the pressure sensor 110 is mounted on the printed circuit board 321; and electrical signal lines are provided. The cover 323 is provided on the printed circuit board 321 to cover the pressure sensor 110. A microphone in which the dynamic range can be enlarged can be provided.

### Fifth Embodiment

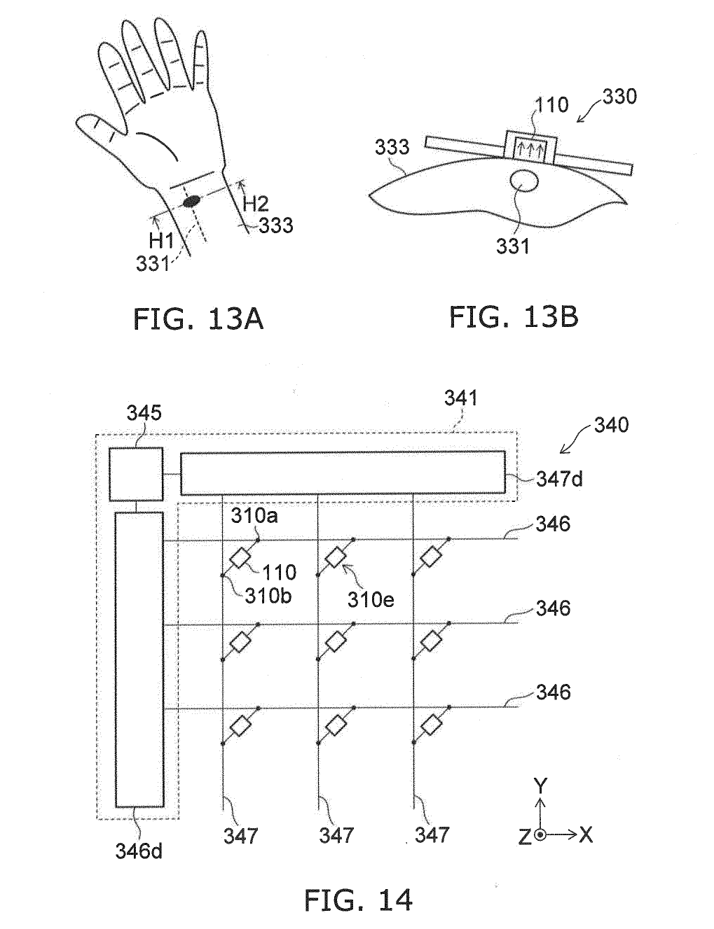

FIG. 13A and FIG. 13B are schematic views illustrating a blood pressure sensor according to a fifth embodiment.

FIG. 13A is a schematic plan view illustrating skin on an arterial vessel of a human. FIG. 13B is a line H1-H2 cross-sectional view of FIG. 13A.

The blood pressure sensor 330 according to the embodiment includes any pressure sensor according to the embodiments or a modification of the embodiments. In the example, the pressure sensor 110 is used as the pressure sensor. The pressure sensor 110 is pressed onto the skin 333 on the arterial vessel 331. Thereby, the blood pressure sensor 330 can continuously perform blood pressure measurements. According to the embodiment, a blood pressure sensor in which the dynamic range can be enlarged can be provided. The blood pressure can be measured with high sensitivity.

### Sixth Embodiment

FIG. 14 is a schematic view illustrating a touch panel according to a sixth embodiment.

The touch panel 340 according to the embodiment includes any pressure sensor according to the embodiments or a modification of the embodiments. In the example, the pressure sensor 110 is used as the pressure sensor. In the touch panel 340, the pressure sensors 110 are mounted to at least one of the interior of the display or the exterior of the display.

For example, the touch panel 340 includes multiple first interconnects 346, multiple second interconnects 347, the multiple pressure sensors 110, and a controller 341.

In the example, the multiple first interconnects 346 are arranged along the Y-axis direction. Each of the multiple first interconnects 346 extends along the X-axis direction. The multiple second interconnects 347 are arranged along the X-axis direction. Each of the multiple second interconnects 347 extends along the Y-axis direction.

The multiple pressure sensors 110 are provided respectively at the crossing portions between the multiple first interconnects 346 and the multiple second interconnects 347. One pressure sensor 110 is used as one sensing component 310e for sensing. Here, the crossing portion includes the position where the first interconnect 346 and the second interconnect 347 cross and includes the region at the periphery of the position.

One end 310.a of each of the multiple pressure sensors 110 is connected respectively to the multiple first interconnects 346. One other end 310b of each of the multiple pressure sensors 110 is connected respectively to the multiple second interconnects 347.

The controller 341 is connected to the multiple first interconnects 346 and the multiple second interconnects 347. For example, the controller 341 includes a first interconnect circuit 346d that is connected to the multiple first interconnects 346, a second interconnect circuit 347d that is connected to the multiple second interconnects 347, and a control circuit 345 that is connected to the first interconnect circuit 346d and the second interconnect circuit 347d. The pressure sensor 110 is compact and can perform highly-sensitive pressure sensing. Therefore, it is possible to realize a high definition touch panel.

According to the embodiment, a touch panel in which the dynamic range can be enlarged can be provided. A highly-sensitive touch input is possible.

Other than the applications recited above, the pressure sensors according to the embodiments are applicable to an atmospheric pressure sensor, an air pressure sensor of a tire, etc. The pressure sensors according to the embodiments are applicable to various pressure sensing.

According to the embodiments, a pressure sensor, a microphone, a blood pressure sensor, and a touch panel in which the dynamic range can be enlarged can be provided.

According to the embodiments, a pressure sensor, a pressure sensor system, a microphone, a blood pressure sensor, and a touch panel can be provided in which the sensitivity can be increased.

Hereinabove, exemplary embodiments of the invention are described with reference to specific examples. However, the embodiments of the invention are not limited to these specific examples. For example, one skilled in the art may similarly practice the invention by appropriately selecting specific configurations of components included in sensors such as film portions, sensor unites, structure bodies, etc., from known art. Such practice is included in the scope of the invention to the extent that similar effects thereto are obtained.

Further, any two or more components of the specific examples may be combined within the extent of technical feasibility and are included in the scope of the invention to the extent that the purport of the invention is included.

Moreover, all pressure sensors, all pressure sensor systems, all microphones, all blood pressure sensors, and all touch panels practicable by an appropriate design modification by one skilled in the art based on the pressure sensors, the pressure sensor systems, the microphones, the blood pressure sensors, and the touch panels described above as embodiments of the invention also are within the scope of the invention to the extent that the spirit of the invention is included.

Various other variations and modifications can be conceived by those skilled in the art within the spirit of the invention, and it is understood that such variations and modifications are also encompassed within the scope of the invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. A pressure sensor, comprising:
a film portion having a front surface, the film portion being deformable;
a first sensor unit separated from a first portion of an outer edge of the front surface and fixed to one portion of the front surface, the first sensor unit including a plurality of first sensing elements arranged along the front surface, one of the plurality of first sensing elements including a first magnetic layer, a first opposing magnetic layer, and a first nonmagnetic intermediate layer provided between the first magnetic layer and the first opposing magnetic layer; and
a first structure body arranged with the first sensor unit along the arrangement direction of the plurality of first sensing elements, the first structure body including a first structure body layer, a first opposing structure body layer, and a first intermediate structure body layer provided between the first structure body layer and the first opposing structure body layer, the first structure body layer having at least one of a floating potential with respect to the first opposing structure body layer or same potential as a potential of the first opposing structure body layer.

2. The sensor according to claim 1, wherein
the first structure body layer includes same material as a material included in the first magnetic layer,
the first opposing structure body layer includes same material as a material included in the first opposing magnetic layer, and
the first intermediate structure body layer includes same material as a material included in the first nonmagnetic intermediate layer.

3. The sensor according to claim 1, wherein
the outer edge includes:
a first side extending in a first direction;
a second side extending in the first direction and being separated from the first side in a second direction, the second direction crossing the first direction;
a third side extending in the second direction; and
a fourth side extending in the second direction and being separated from the third side in the first direction,
the plurality of first sensing elements is arranged in the first direction of the first side, and
the first structure body is provided between the third side and one end of the first sensor unit.

4. The sensor according to claim 3, wherein a distance along the first direction between the first side and the second side is longer than a distance along the second direction between the third side and the fourth side.

5. The sensor according to claim 1, wherein
the first structure body further includes:
a first conductive layer electrically connected to the first structure body layer; and
a second conductive layer electrically connected to the first opposing structure body layer,
the first structure body layer is provided between the first conductive layer and the second conductive layer, and
the first opposing structure body layer is provided between the first structure body layer and the second conductive layer.

6. The sensor according to claim 5, wherein the first structure body further includes an interconnect layer electrically connecting the first conductive layer and the second conductive layer.

7. The pressure sensor according to claim 5, wherein the first sensor unit further includes:
a first electrode electrically connected to the first magnetic layer; and
a second electrode electrically connected to the first opposing magnetic layer,
the first magnetic layer is provided between the first electrode and the second electrode, and
the first opposing magnetic layer is provided between the first magnetic layer and the second electrode.

8. The sensor according to claim 7, wherein
the first conductive layer includes a material included in the first electrode, and
the second conductive layer includes a material included in the second electrode.

9. The sensor according to claim 3, further comprising a second structure body arranged with the first sensor unit along the first direction and provided between the fourth side and one other end of the first sensor unit,
the second structure body including a second structure body layer, a second opposing structure body layer, and a second intermediate structure body layer provided between the second structure body layer and the second opposing structure body layer,
the second structure body layer having at least one of a floating potential with respect to the second opposing structure body layer or same potential as a potential of the second opposing structure body layer.

10. The sensor according to claim 3, further comprising:
a second sensor unit including a plurality of second sensing elements, the plurality of second sensing elements being arranged in the first direction of the second side, one of the plurality of second sensing elements including a second magnetic layer, a second opposing magnetic layer, and a second nonmagnetic intermediate layer provided between the second magnetic layer and the second opposing magnetic layer; and
a third structure body arranged with the second sensor unit along the first direction and provided between the fourth side and one end of the second sensor unit, the third structure body including a third structure body layer, a third opposing structure body layer, and a third intermediate structure body layer provided between the third structure body layer and the third opposing structure body layer, the third structure body layer having at least one of a floating potential with respect to the third opposing structure body layer or same potential as a potential of the third opposing structure body layer.

11. The sensor according to claim 10, further comprising a fourth structure body arranged with the second sensor unit along the first direction and provided between the third side and one other end of the second sensor unit,
the fourth structure body including a fourth structure body layer, a fourth opposing structure body layer, and a fourth intermediate structure body layer provided between the fourth structure body layer and the fourth opposing structure body layer,
the fourth structure body layer having at least one of a floating potential with respect to the fourth opposing structure body layer or same potential as a potential of the fourth opposing structure body layer.

12. The sensor according to claim 3, further comprising:
a fifth structure body provided between the first structure body and the third side; and
a sixth structure body provided between the second structure body and the fourth side,
the fifth structure body including a fifth structure body layer, a fifth opposing structure body layer, and a fifth intermediate structure body layer provided between the fifth structure body layer and the fifth opposing structure body layer,
the sixth structure body including a sixth structure body layer, a sixth opposing structure body layer, and a sixth intermediate structure body layer provided between the sixth structure body layer and the sixth opposing structure body layer,
the fifth structure body layer having at least one of a floating potential with respect to the fifth opposing structure body layer or same potential as a potential of the fifth opposing structure body layer,
the sixth structure body layer having at least one of a floating potential with respect to the sixth opposing structure body layer or same potential as a potential of the sixth opposing structure body layer.

13. The pressure sensor according to claim 1, wherein
the first magnetic layer includes at least one of Fe, Co, or Ni, and
the first opposing magnetic layer includes at least one of Fe, Co, or Ni.

14. The sensor according to claim 1, wherein the plurality of first sensing elements is connected in series.

15. The sensor according to claim 1, further comprising a holder holding the film portion.

16. The pressure sensor according to claim 1, wherein a magnetization of the first magnetic layer changes according to a deformation of the film portion.

17. A pressure sensor, comprising:
a film portion having a front surface, the film portion being deformable;
a first sensor unit separated from a first portion of an outer edge of the front surface and fixed to one portion of the front surface, the first sensor unit including a plurality of first sensing elements arranged along the front surface, one of the plurality of first sensing elements including a first magnetic layer, a first opposing magnetic layer, and a first nonmagnetic intermediate layer provided between the first magnetic layer and the first opposing magnetic layer; and
a first structure body arranged with the first sensor unit along the arrangement direction of the plurality of first sensing elements, the first structure body including a first structure body layer, a first opposing structure body layer, and a first intermediate structure body layer provided between the first structure body layer and the first opposing structure body layer, the first structure body layer having at least one of a floating potential with respect to the first opposing structure body layer or same potential as a potential of the first opposing structure body layer,
the first sensor unit including:
a first electrode electrically connected to the first magnetic layer; and
a second electrode electrically connected to the first opposing magnetic layer,
the first magnetic layer being provided between the first electrode and the second electrode, and
the first opposing magnetic layer being provided between the first magnetic layer and the second electrode,
the film portion including:
a first region overlapping the first electrode, the first magnetic layer and the second electrode,
a second region not overlapping the first electrode, the first magnetic layer and the second electrode,
a third region overlapping the first electrode, the first magnetic layer and the second electrode, and
a fourth region not overlapping the first electrode, the first magnetic layer and the second electrode,
the second region being located between the first region and the third region, and
the third region being located between the second region and the fourth region.

18. A pressure sensor system, comprising:
a film portion having a front surface, the film portion being deformable;
a first sensor unit separated from a first portion of an outer edge of the front surface and fixed to one portion of the front surface, the first sensor unit including a plurality of first sensing elements arranged along the front surface, one of the plurality of first sensing elements including a first magnetic layer, a first opposing magnetic layer, and a first nonmagnetic intermediate layer provided between the first magnetic layer and the first opposing magnetic layer;
a first structure body arranged with the first sensor unit along the arrangement direction of the plurality of first sensing elements, the first structure body including a first structure body layer, a first opposing structure body layer, and a first intermediate structure body layer provided between the first structure body layer and the first opposing structure body layer; and
a controller connected with the first sensor unit and the first structure body,
the controller being configured to supply a current to the first sensor unit,
the controller being configured to electrically connect the first structure body layer with the first opposing structure body layer, or to make an electrical potential of the first structure body layer floating with respect to an electrical potential of the first opposing structure body.

19. A microphone, comprising
the pressure sensor according to claim 1.

20. A blood pressure sensor, comprising
the pressure sensor according to claim 1.
